(19) United States
(12) Patent Application Publication
(10) Pub. No.: US 2017/0209039 A1
(43) Pub. Date: Jul. 27, 2017

(54) METHOD FOR TESTING VISUAL ACUITY

(71) Applicant: OBSCHESTVO S OGRANICHENNOJ OTVETSTVENNOST'JU "OST-OPTIK SK", Vladivostok (RU)

(72) Inventor: Aleksandr Ivanovich MYAGKIKH, Vladivostok (RU)

(21) Appl. No.: 15/314,023

(22) PCT Filed: May 27, 2014
(86) PCT No.: PCT/RU2014/000388

§ 371 Date: Nov. 25, 2016

(51) Int. Cl. A61B 3/028; A61B 3/02; A61B 3/032; A61B 3/00

(52) U.S. Cl. CPC A61B 3/028; A61B 3/0033; A61B 3/022; A61B 3/032

(57) ABSTRACT

The invention relates to the field of medicine, and more specifically to ophthalmology. The technical aim consists in increasing the accuracy of determining the visual acuity of patients who simulate higher (for instance, when applying for a job) or lower (for instance, when registering a disability) visual acuity. The technical aim is achieved by means of a method for determining visual acuity which involves showing to a patient test symbols on a display, the size of which symbols is continuously adjustable over a wide dynamic range. As the size of the optotypes is adjusted, the orientation of said optotypes is periodically changed or certain optotypes are replaced with other optotypes of the same size. Visual acuity is determined on the basis of computing an angle at which an optotypes of a minimum distinguishable size can be seen, and a visual acuity value is registered and recorded. This procedure is repeated a number of times, a mean value is determined for the obtained visual acuity data, a relative standard deviation is calculated and if the value thereof exceeds 5%, it is concluded that the measurements performed were not sufficiently accurate and/or that the patient was simulating and that measures must be taken to correct the test conditions and/or to eliminate patient simulation by requiring a repetition of the test.

### CROSS-REFERENCE

The present application is a National Phase Entry of International Patent Application no. PCT/RU2014/000388, filed on May 27, 2015, entitled “METHOD FOR TESTING VISUAL ACUITY”. This application is incorporated by reference herein in its entirety.

### FIELD OF THE INVENTION

The invention relates to the field of medicine, and more specifically, to ophthalmology.

### BACKGROUND

The acuity characterizes the eye's ability to distinguish fine details of the image. To be specific, it is considered that this is equivalent to discriminate of two points separated by a small distance. Visual acuity in this representation is characterized by a minimum angular distance at which the points are seen separately (see http://dic.academic.ru/dic.nsf/enc_{—medicine/}21507/). Numerically, visual acuity is expressed in the form of a reverse angle expressed in minutes of arc angle. For example, the angle discrimination in one angular minute corresponds to a visual acuity of 1.0; the angle discrimination in two angular minutes corresponds to visual acuity 0.5.

Methods are known for determining the visual acuity of the patient based on the presentation of chart containing a set of training symbols (optotypes) of different size arranged in rows. The size of the characters in each row corresponds to a specific visual acuity when observing table from the standard distance. The tabular presentation method is not a principled (paper table lighting, projector, screen display).

The known methods differ from each other in the content of the charts and the distance, with whose visual acuity is determined (see: “About the systems and the rules of visual acuity determination” I. A. Leshchenko “Modern optometry”, 2009, # 3, pp. 54-58.; http://www.optica4all.ru/files/spravuchmat/Public/VA.pdf).

The Snellen or Golovin-Sivtsev charts (12 lines), inserted in the device with a lighter (Roth apparatus), are most commonly used in ophthalmological practice.

The disadvantages of the known methods for determining vision acuity should be considered:

1. The limited range of visual acuity, since any chart has maximum and minimum size of the test symbol. For example, in Russia the routine Golovin-Sivtsev's chart designed to determine visual acuity in the range from 0.1 to 2.0.
2. Low accuracy in determining visual acuity determines by the number of rows in the chart. For example, for Golovin-Sivtsev's chart in the range from 0.1 to 1.0 the number of lines is 10, therefore, the relative accuracy of visual acuity determination in the field of low acuity (0.1 to 0.2), will be about 50%; in a region of high acuity (0.9 to 1.0)—about 10%.

These disadvantages become critical during the study of dynamics of visual acuity during treatment or vision correction. In particular, the determination of visual acuity plays a significant role during refractive surgery: when patient's visual acuity before operation is much lower than 0.1 and after operation is higher than 1.0. The accuracy of the visual acuity is especially important if the numerical values must be carried out with mathematical operations. For example: in the case of determining the efficacy of treatment. Since the change of the visual acuity values before and after treatment may be correctly compared only in the form of their ratio, inaccuracy determining each of numbers will lead to unacceptable uncertainty in the result.

A device is known for testing visual acuity (Russian Patent for utility model no. 73186) main technical characteristic of which is the possibility of submitting the patient to test optotypes, which size is smoothly (with high discreteness) varies over a wide dynamic range. This significantly improves the range and accuracy of vision acuity single count.

The disadvantage of determining visual acuity using the present device is the uncertainty of the degree of accuracy of a single reference, as a consequence, the difficulty of determining the true visual acuity of patient, who tries to enter doctor misleading by guessing optotypes or, on the contrary, pretending that does not distinguish between them.

Technical problem: increase of accuracy of visual acuity determination for patients, simulating raised (e.g., for employment) or lower (e.g., for registration of disability) visual acuity.

The technical problem is solved by a method for determining visual acuity, which consists in the presentation of the patient displayed on the test characters, the size of which continuously adjustable over a wide dynamic range, and the determination of visual acuity on the basis of calculating the angle at which an optotypes of a minimum distinguishable size can be seen during resizing optotypes periodically change their orientation or substitute some other optotypes of the same size, thus establishing the minimum discernible size optotypes that the patient confidently identify, fix and store value of visual acuity, a mean value is determined for the obtained visual acuity data, a relative standard deviation is calculated and, depending on this value, make conclusion about the presence or absence of simulation (conscious malingering) and, in the absence of such—about the accuracy of the measurement carried out.

The totality of the essential distinctive features, claimed by author, is necessary and sufficient to uniquely solve the problem.

The inventor conducted extensive work to determine the intervals of the main parameters. The dynamic range of optotypes imaging is determined as the ratio of the maximum size to the minimum size of the reproduced optotypes. The maximum size corresponds to optotype's dimension, when it fully inscribed in the aperture. The minimum size—when it is inscribed in the area of 5×5 pixels of the display. For modern computer displays (monitors) the dynamic range of the displayed dimensions optotypes will exceed 200 (compared with 20 in known methods and devices). The same number (200) will determine the lower limit of discreteness of optotype's sizes and thus theoretically attainable accuracy of determining visual acuity. A recommended image distance optotypes is 5 meters. At such distance a standard 22-inch display with a resolution of 1680 ×1050 allows the determination of visual acuity from 0.027 to 5.57, against 0.1-2.0 in a known method (The Eye, 2011, #. 3, pp. 35-37).

The relative accuracy of visual acuity determining is directly related to the accuracy of establishing the size of test optotypes, and in this case will not exceed 0.5% of the area of low visual acuity (0.1-0.2) and not more than 4% in the area of high acuity (0.9-1.0).

The method is carried out as follows.

At the beginning of the procedure of visual acuity determining patient from a certain distance are brought on average size of optotypes (the middle of the scale size change), that appear on the computer managed display. Depending on whether or not the patient identifies them, the optotypes is continuously adjustable (reduction/enlargement) with simultaneous periodic change of their orientation or substitute them with other optotypes of the same size, establishing their smallest distinguishable dimension, and then by a known distance to the image size and installed size of optotypes, visual acuity value is calculated and stored. The procedure is repeated several times (from 3 to 5). Then determine the average value of visual acuity and taking it as target value (PV Novitsky, I A Zograf, “The error estimation of measurement results”,-2-th Edition, Revised and Enlarged, L.: Energoatomizdat. Leningrad's Div., 1991, P. 141). Further, obtained series of visual acuity values used for calculating the relative standard deviation (also called as “variation coefficient”) and expressed it in percentage (GOST R 50779.10-2000 <<Statistical methods. Probability and statistics basics>>, item 1.24, http://docs.cntd.ru/document/gost-r-50779-10-2000). The relative standard deviation determines the accuracy of the measurement has been carried out. If the relative standard deviation exceeds 5%, this session should be recognized as unsatisfactory, requiring repetition (refinement), because the accuracy of such measurements is worse than 10% provided by conventional devices of visual acuity determination.

The accuracy of visual acuity determination compared to the known device (Russian Patent for utility model no. 73186) increases by performing several measurements, and calculating the average value, since this procedure entails a reduction of result's random errors. This decrease is proportionally to the square root of the number of independent measurements. Thus, for the five samples the random error in determining reduced by more than two times (PV Novitsky, I A Zograf, “The error estimation of measurement results”, -2-th Edition, Revised and Enlarged, L.: Energoatomizdat. Leningrad's Div., 1991, P. 142).

The resulting vision acuity is recorded as the “mean value plus or minus the relative standard deviation”.

The practice of measuring visual acuity indicates that attempts to defraud the physician by patient consist in guessing of unrecognized optotypes, or ignoring the well recognized optotypes, inevitably results in considerable scatter of visual acuity values. The experience of visual acuity testing using this technique in “Ost-Optik K” Co., Ltd indicates that in the case of unsatisfactory test conditions and/or simulation of the patient the relative standard deviation will accept values larger than 5%. Such measurement sessions must be considered unsatisfactory. Accordingly, measures should be taken to remedy the testing conditions or patient should be required to terminate the sham.

The totality of the essential distinctive features of the invention specified in the claims provides a unique positive solution to the stated technical problem. Compared with the known methods author managed to reliably capture cases of simulation and, considering this, to increase the accuracy of the determined true visual acuity.

Examples of visual acuity tests, using this method

| | Patient | Patient | Patient | Patient | Patient | Patient |
|---|---|---|---|---|---|---|
| | 1 | 2 | 3 | 4 | 5 | 6 |
| Sample 1 | 1.3 | 1.05 | 0.77 | 0.34 | 0.1 | 0.055 |
| Sample 2 | 1.33 | 1.07 | 0.74 | 0.35 | 0.11 | 0.06 |
| Sample 3 | 1.35 | 1.04 | 0.75 | 0.33 | 0.13 | 0.054 |
| Sample 4 | 1.29 | 1.08 | 0.86 | 0.34 | 0.09 | 0.057 |
| Sample 5 | 1.33 | 1.05 | 0.79 | 0.34 | 0.12 | 0.06 |
| Mean Value | 1.3198 | 1.0579 | 0.7809 | 0.3399 | 0.1091 | 0.0571 |
| Standard | 0.0219 | 0.0147 | 0.0426 | 0.0063 | 0.0141 | 0.0025 |
| Deviation | | | | | | |
| Relative | 0.0166 | 0.0139 | 0.0546 | 0.0186 | 0.1296 | 0.0434 |
| Standard | | | | | | |
| Deviation | | | | | | |
| Visual | 1.320 | 1.058 | 0.781 | 0.340 | 0.109 | 0.057 |
| Acuity | | | | | | |
| Plus-minus | 1.7% | 1.4% | 5.5% | 1.9% | 13.0% | 4.3% |
| Reliability | Yes | Yes | No | Yes | No | Yes |
| Conclusion: | Approve | Approve | Re- | Approve | Simu- | Approve |
| | | | check | | lation | |

1. A method of the visual acuity testing, comprising presentation to the patient on display the training symbols, the size of which is changed with high granularity in a wide dynamic range, during resizing optotypes periodically change their orientation or replace by others of the same size, define the visual acuity based on calculating the angle at which the optotypes has minimum discernable visible size, fix and store the value of the visual acuity, characterized in that the procedure is repeated several times, calculated the average value of visual acuity data and the relative standard deviation, and if its value exceeds 5%, a conclusion is drawn about unsatisfactory accuracy of the results of conducted visual acuity measurement, and patient simulation should be deleted by the repetition of research.
